(19) United States
(12) Patent Application Publication
(10) Pub. No.: US 2020/0261348 A1
(43) Pub. Date: Aug. 20, 2020

(54) SILOXANE ELASTOMERS HAVING AN IMMEDIATE OPTICAL AND SENSORIAL SMOOTHING EFFECT AND TOPICAL SKIN-CARE COMPOSITIONS CONTAINING SUCH SILICONE ELASTOMERS

(71) Applicant: BEIERSDORF AG, Hamburg (DE)

(72) Inventors: Julia PRUNS, Hamburg (DE); Lisa PESCHKE, Hamburg (DE); Thomas RASCHKE, Pinneberg (DE); Imke KUHLMANN, Hamburg (DE)

(21) Appl. No.: 16/061,399

(22) PCT Filed: Nov. 25, 2016
(86) PCT No.: PCT/EP2016/078819

§ 371 Date: Jun. 12, 2018

(30) Foreign Application Priority Data
Dec. 21, 2015 (DE) 102015226221.9

(51) Int. Cl. A61K 8/895; C08L 83/04; C08K 5/56

(52) U.S. Cl. CPC A61K 8/895; C08G 77/08; C08K 5/56; C08L 83/04

(57) ABSTRACT

The invention relates to siloxane elastomers, which can be obtained by the reaction of A one or more oligomeric organohydrogensiloxanes having at least two SiH-bearing siloxane units per molecule with B one or more oligomeric divinylsilanes and the use of C platinum(0)-1,2-divinyl-1,1,3,3-tetramethyldisiloxane as a hydrosilylation catalyst.

The invention relates to siloxane elastomers and also to topical skincare compositions containing such silicone elastomers and also to methods for the preparation thereof and the use thereof.

Many personal care products currently available to consumers are primarily aimed at improving the health or the external appearance of the skin or of the hair. Among skincare products, many are aimed at delaying, minimizing or even eliminating the formation of skin wrinkles and other histological changes which are generally associated with skin aging or with skin damage due to environmental effects.

Numerous compounds have been described in the prior art as being suitable for controlling the state of the skin, including controlling fine lines, wrinkles and other forms of uneven or rough surface quality associated with aged or photodamaged skin.

The skin is subject to stresses from numerous external and internal factors. Extrinsic factors include ultraviolet radiation (e.g. solar radiation), environmental pollution, wind, heat, low humidity, aggressive surfactants, abrasives and the like.

Intrinsic factors include chronological age and other biochemical changes within the skin. Whether these factors are external or internal, they result in visible signs of skin aging and damage from environmental effects, such as the appearance of wrinkles and other forms of roughness (including enlarged pores, flaky skin and skin lines) and also other histological changes associated with skin aging or skin damage. Skin wrinkles remind many of the loss of youth.

A further aim of skincare is to compensate for the loss of oils and water from the skin caused by daily washing. This becomes particularly important if the natural capacity for regeneration is insufficient. In addition, skincare products should protect against environmental effects, in particular sun and wind, and delay skin aging.

Chronological skin aging is caused for example by endogenous, genetically determined factors. In the epidermis and dermis, age-related process result, for example, in the following structural damage and functional disruptions which can also be grouped together under the term “senile xerosis”:

a) dryness, unevenness and formation of fine wrinkles caused by dryness,

b) itching and

c) decreased refatting by sebaceous glands (for example after washing).

Exogenous factors, such as UV light and chemical toxins, may have a cumulative effect and for example accelerate or supplement the endogenous aging process. In the epidermis and dermis, exogenous factors result, for example, in the following structural damage and functional disruptions in the skin, which surpass the damage from chronological aging in both quality and quantity:

d) visible vascular dilations (telangiectases, couperosis);

e) slackness and formation of wrinkles;

f) local instances of hyper-, hypo- and dyspigmentation (for example age spots), and

g) increased susceptibility to mechanical stress (for example cracking).

The present invention relates especially to products for caring for naturally aged skin and also for treating the secondary damage from photoaging, in particular the phenomena mentioned under a) to g).

Skin unevenness, for example small wrinkles, is a particularly undesirable phenomenon accompanying skin aging. One of the aims of cosmetic compositions is to either do away with, override and/or minimize these phenomena. One way to do this consists in supplementing the skin with care components or active ingredients, but this can represent a protracted undertaking.

Products for caring for aged skin contain, for example, retinoids (vitamin A acid and/or derivatives thereof) or vitamin A and/or derivatives thereof. However, their effect on structural damage is limited in terms of scope. Moreover, during product development there are considerable difficulties in stabilizing the active ingredients sufficiently against oxidative degradation. Moreover, the use of products containing vitamin A acid often causes skin irritation. Therefore, retinoids can only be used in low concentrations.

In this context, it is desirable to have a visible effect immediately after application of the cosmetic composition, but at the same time avoiding a “mask-like” impression of the skin area in question.

It is known that the use of “soft-focus” raw materials can optically conceal relatively small instances of skin unevenness and fine wrinkles. Such materials are notable for suitable light refraction and light scattering, and soften the surface appearance of the skin through their blurring effect.

Siloxane elastomers are used in cosmetic preparations due to their silky and powdery sensory properties. Siloxane elastomers are three-dimensionally crosslinked silicone polymers. In contrast to non-crosslinked silicone polymers, they have different properties to these. Although they often contain more than 80% of included silicone oil, they usually do not cause greasiness, but rather on the contrary cause a soft or even powdery skin feel. Silicone elastomers are also proposed as “soft-focus” raw materials.

U.S. Pat. No. 4,256,870 describes crosslinked siloxane polymers with the use of a vinylsilane and a hydrosilane.

EP 381 166 and EP 383 540 describe the principle of the reaction of a hydrosilane and a vinylsilane with platinum catalysis and the use of the siloxane elastomers obtained in cosmetic preparations.

It was an object of the invention to provide novel silicone elastomers with improved cosmetic properties.

It was a further object of the invention to provide cosmetic preparations which, on the one hand, afford broad possibilities for use, and, on the other hand, ensure as natural an impression as possible of the appearance of the skin after application, and at the same time reduce or even conceal entirely the presence of skin unevenness, in particular of fine wrinkles.

These objects are achieved by siloxane elastomers obtainable by carrying out the reaction of

A one or more oligomeric organohydrosiloxanes having at least two SiH-bearing siloxane units per molecule of the following structure

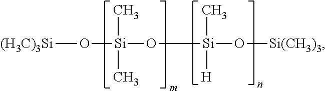

wherein m=0-40, preferably 0-30, especially preferably 0-20, and
n=2-25 preferably 2-15, especially preferably 4-10, and

B one or more oligomeric divinylsilanes of the following structure

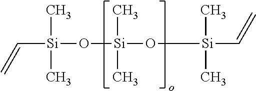

with o=5-90, preferably 10-80 and particularly preferably 15-70,

with use of

C platinum(0)-1,2-divinyl-1,1,3,3-tetramethyldisiloxane which is characterized by the structure

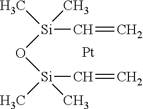

as hydrosilylation catalyst, wherein the divinylsilane is dissolved in a silicone oil (advantageously dimethicone) and is heated to temperatures in the range from 25-45° C. with stirring, and wherein, after the catalyst has been stirred in, the organohydrosiloxane is added.

The reaction begins immediately and is concluded as soon as the consistency in the entire reaction vessel has increased, and does not increase further.

Subsequently, the polymer is sheared in a controlled manner with addition of further solvent. This is carried out using a rotor-stator stirring system.

Subsequently, the polymer is sheared in a controlled manner with addition of further solvent. This is carried out using a rotor-stator stirring system (e.g. “L5M-A” from Silverson).

For this purpose, an amount of the crosslinked silicone polymer is added to a suitable vessel with an additional amount of silicone oil (e.g. dimethicone) and sheared for a defined length of time using the stirring system. This length of time is very heavily dependent on the size of the batch to be sheared. On the one hand, the length of time must be sufficient to completely shear the crosslinking product (and not only a portion thereof). On the other hand, the length of time selected must not be so long that too much shearing potentially destroys the polymer and limits the three-dimensional crosslinking. In this case, the weights of polymer and silicone oil are selected such that a polymer concentration of 5 to 20% is achieved. A polymer concentration of 15-20% has proven to be particularly advantageous.

For a batch of 1 kg, for example, one minute using the L5M-A high-shear mixer from Silverson (using the “multi-purpose comminuting stator” and a rotary speed of 8000 rpm) achieves suitable shearing. It is important to ensure uniform shearing of the whole product. Depending on the size of the reaction system, the duration of shearing may vary considerably from one minute up to 15 min or more.

The inventive cosmetic formulation also includes cosmetic preparations having a content of such silicone elastomers.

The invention also includes a method for preparing silicone elastomers which is characterized in that

A one or more oligomeric organohydrosiloxanes having at least two SiH-bearing siloxane units per molecule of the following structure

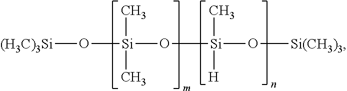

wherein m=0-40, preferably 0-30, especially preferably 0-20, and
n=2-25 preferably 2-15, especially preferably 4-10, and

B one or more oligomeric divinylsilanes of the following structure

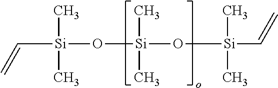

with o=5-90, preferably 10-80 and particularly preferably 15-70,

with use of

C platinum(0)-1,2-divinyl-1,1,3,3-tetramethyldisiloxane which is characterized by the structure

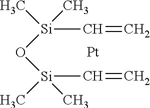

as hydrosilylation catalyst, wherein the divinylsilane is dissolved in a silicone oil (advantageously dimethicone) and is heated to temperatures in the range from 20-60° C., preferably 25-45° C., with stirring, and wherein, after the catalyst has been stirred in, the organohydrosiloxane is added.

Advantageously, the molar ratios of A to B are selected from the range from 10:1 to 1:10, preferably from 5:1 to 1:5, especially preferably from 2:1 to 1:2.

After the catalyst has been stirred in, the organohydrosiloxanes are added. The reaction begins immediately and is concluded as soon as the consistency in the entire reaction vessel has increased.

Subsequently, the polymer is sheared in a controlled manner with addition of further solvent. This is carried out using a rotor-stator stirring system (e.g. L5M-A from Silverson).

For this purpose, an amount of the crosslinked silicone polymer is added to a suitable vessel with an additional amount of silicone oil (e.g. dimethicone) and sheared for a defined length of time using the stirring system. This length of time is very heavily dependent on the size of the batch to be sheared. On the one hand, the length of time must be sufficient to completely shear the crosslinking product (and not only a portion thereof). On the other hand, the length of time selected must not be so long that too much shearing potentially destroys the polymer and limits the three-dimensional crosslinking. In this case, the weights of polymer and silicone oil are selected such that a polymer concentration of 5 to 20% is achieved. A polymer concentration of 15-20% has proven to be particularly advantageous.

For a batch of 1 kg, for example, one minute using the high-shear mixer L5M-A from Silverson (using the “multi-purpose comminuting stator” and a rotary speed of 8000 rpm) achieves suitable shearing. It is important to ensure uniform shearing of the whole product. Depending on the size of the reaction system, the duration of shearing may vary considerably from one minute up to 15 min or more.

The inventive silicone elastomers are notable for an enhanced “soft-focus” effect, by means of which relatively minor instances of skin unevenness and small wrinkles can be optically concealed, which will be corroborated below.

For verification of the soft-focus effect, a measurement of the total and scattered transmittance and also the total and scattered reflection is carried out (by means of photometer with Ulbricht sphere). According to Emmert (Cosm. Toil., 111, 1996, p. 57-61), the soft-focus effect is best achieved if as high a proportion of diffuse (i.e. scattered) transmittance as possible, and a high total transmittance, are achieved.

The high transmittance at the same time means low direct reflection, such that shiny skin is avoided and the natural skin tone shows through.

The inventive composition and the raw materials to be compared were spread on a microscope slide as a 30 μm-thick film and were measured after 3 minutes' drying time by means of a double beam photometer (SPECORD 250 UV/VIS spectrophotometer), with use of an Ulbricht sphere (Analytik Jena AG).

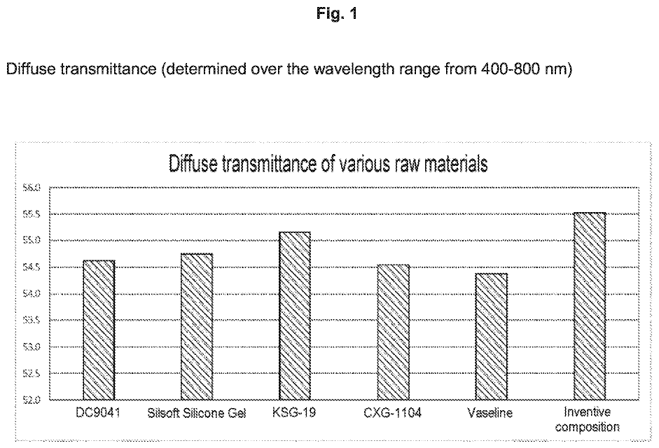

The proportion of diffuse transmittance is valued particularly highly, since it is this which conceals instances of skin unevenness and causes the natural skin tone to show through. The values shown here are mean values across the entire wavelength range of visible light (400-800 nm). See FIG. 1.

The following are measured as comparative specimens:

1. DC 9041 (Dow Corning)

2. Silsoft Silicone Gel (Momentive)

3. KSG-19 (Shin Etsu)

4. CXG-1104 (Nusil)

5. Vaseline (Sasol Wax, negative control)

6. Inventive composition

It is clear here that the inventive composition has the highest proportion of diffuse transmittance.

Furthermore, inventive siloxane elastomers have a particularly tack-reducing effect on the skin. The tack was reduced by means of formulations with high quantities of photoprotective filters, especially water-soluble photoprotective filters. The tack of these formulations was carried out by means of a measurement of “in-vitro sand adhesion”.

In-Vitro Sand Adhesion:

In each case 50 mg of the emulsions to be tested were applied to PMMA Schonberg plaques (5.0×5.0 cm) and spread as uniformly as possible over the plaque, using a fingerstall. The example formula applied is then dried at room temperature for 15 minutes. After this time, the weight of the dried plaques was determined using an analytical balance. Subsequently, the plaques were sprinkled with fine sea sand (extrapure, from Merck KGaA, Darmstadt). Loosely adhering sand was removed by causing the plaques to slide once along a slide apparatus intended for that purpose.

The adhering sand remaining on the plaque thereafter was determined by reweighing. The sand adhesion can be determined using the following equation:

Δ(adhesion) [mg]=m(plaque with sand)[mg]−m(plaque with cream applied)[mg]

The experiments were repeated 10 times per example formula and the results were averaged.

Example formula 6: formula without addition of silicones

Example formula 7: formula with addition of 10% Dimethicone

Example formula 8: formula with addition of 10% Dimethicone/Dimethicone Crosspolymer (DC 9041, Dow Corning)

Example formula 9: formula with addition of 10% Dimethicone/Vinyl Dimethicone Crosspolymer (KSG-19, Shin-Etsu)

Example formula 10: formula with addition of 10% Dimethicone/Vinyl Dimethicone Crosspolymer (inventive composition).

The precise compositions are given in Table 4.

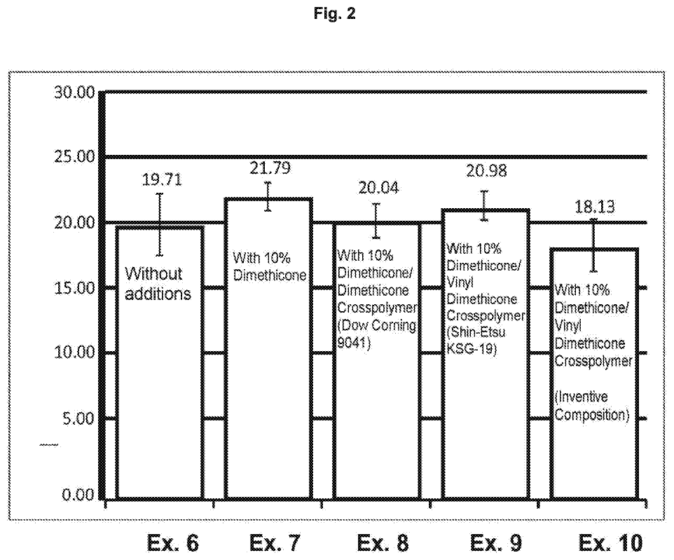

The results of the measurements are summarized in FIG. 2. The inventive composition (Ex. 10) enabled a reduction in the quantity of adhered sand (and hence the tack) compared to the other formulas.

Cosmetic or dermatological preparations according to the invention preferably comprise 0.1-60 wt %, preferably 1-51 wt %, particularly preferably 3-40 wt %, of one or more inventive silicone elastomers based on the total composition of the preparations.

It may also be advantageous to add 0.1-50 wt %, particularly preferably 1-30 wt %, based on the total composition of the preparations, of one or more silicone elastomers of an origin other than the invention to cosmetic or dermatological preparations according to the invention.

Emulsions are advantageous presentation forms in the context of the present invention, for example are advantageous in the form of a cream, a lotion, a cosmetic milk, and contain for example fats, oils, waxes and/or other fatty substances, and also water and one or more emulsifiers as are customarily used for such a type of formulation.

Medicinal topical compositions in the context of the present invention generally comprise one or more medicaments at an effective concentration. For the sake of simplicity, in order to make a clear distinction between cosmetic and medicinal use and corresponding products, reference is made to the statutory provisions of the Federal Republic of Germany (e.g. Kosmetikverordnung [cosmetics directive], Lebensmittel- and Arzneimittelgesetz [food and medicinal products act]).

It is likewise advantageous here to add the active ingredient used according to the invention as additional ingredient to preparations which already comprise other active ingredients for other purposes.

If the cosmetic or dermatological preparation in the context of the present invention is a solution or emulsion or dispersion, it is possible to use, as solvents:
water or aqueous solutions
oils such as capric or caprylic acid triglycerides
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and also the ethers thereof, preferably ethanol, isopropanol, propylene glycol, glycerol, hexanediol, octanediol, ethylhexyl glycerol, glyceryl caprylate, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water may be a further constituent.

The oil phase of the emulsions, oleogels or hydrodispersions or lipodispersions in the context of the present invention is advantageously selected from the group of esters of saturated and/or unsaturated, branched and/or unbranched alkanecarboxylic acids having a chain length from 3 to 30 C atoms and saturated and/or unsaturated, branched and/or unbranched alcohols having a chain length from 3 to 30 C atoms, from the group of esters of aromatic carboxylic acids and saturated and/or unsaturated, branched and/or unbranched alcohols having a chain length of 3 to 30 C atoms. Such ester oils may then advantageously be selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and also synthetic, semisynthetic and natural mixtures of such esters, for example jojoba oil.

Further, the oil phases may advantageously be selected from the group of branched and unbranched hydrocarbons and hydrocarbon waxes, silicone oils, dialkyl ethers, the group of saturated or unsaturated, branched or unbranched alcohols, and also fatty acid triglycerides, namely triglyceryl esters of saturated and/or unsaturated, branched and/or unbranched alkanecarboxylic acids having a chain length of 8 to 24, especially 12 to 18 C atoms. The fatty acid triglycerides may for example advantageously be selected from the group of synthetic, semisynthetic and natural oils, e.g. olive oil, sunflower oil, soybean oil, groundnut oil, rapeseed oil, almond oil, palm oil, coconut oil, palm kernel oil, and other similar oils.

Any blends of such oil and wax components are also advantageous.

The aqueous phase of the inventive preparations advantageously optionally comprises one or more thickeners which may advantageously be selected from the group of silicon dioxide, aluminum silicates, polysaccharides and derivatives thereof, e.g. hyaluronic acid, xanthan gum, cellulose ethers such as, for example, hydroxypropyl methylcellulose, particularly advantageously from the group of polyacrylates, preferably a polyacrylate from the group of the “carbopols”, for example carbopols of 980, 981, 1382, 2984, 5984, Ultrez 10 or ETD 2020 type, in each case individually or in combination.

Solid sticks comprise for example natural or synthetic waxes, fatty alcohols or fatty acid esters.

Customary base materials which are suitable for use as cosmetic sticks in the context of the present invention are liquid oils (e.g. paraffin oils, castor oil, isopropyl myristate), semi-solid constituents (e.g. vaseline, lanolin), solid constituents (e.g. beeswax, ceresin and microcrystalline waxes or ozokerite) and also high-melting point waxes (e.g. carnauba wax, candelila wax).

Suitable as propellant for cosmetic and/or dermatological preparations in the context of the present invention that are sprayable from aerosol containers are the readily volatile, liquefied propellants that are customary and known, for example hydrocarbons (propane, butane, isobutane), which can be used alone or in a mixture with one another. Compressed air may also be advantageously used.

The examples below are intended to illustrate the present invention without limiting it. Unless otherwise stated, all quantitative data, fractions and percentages are based on the weight and the total amount or on the total weight of the preparations.

### EXAMPLE 1

### Preparation of Elastomer with Particularly Potent Optical Effects

The siloxane elastomer with particularly potent optical effects is prepared as described below:

A Two organohydrosiloxanes (e.g. Silmer H D0 (denoted H1) and Silmer H J2 (denoted H2) (Siltech LLC, Lawrenceville, Ga., U.S.A.)) are reacted with

B Two divinylsilanes (e.g. Silmer Vin 15 (denoted V1) and Silmer Vin 70 (denoted V2) (Siltech LLC, Lawrenceville, Ga., U.S.A.)) using the catalyst

C Platinum(0)-1,2-divinyl-1,1,3,3-tetramethyldisiloxane (e.g. as solution in polydimethylsiloxane (Sigma-Aldrich GmbH, Munich, Germany))

in the following manner, in dimethicone as solvent. The quantities of the starting materials are selected such that the molar quantity of silane groups corresponds to the molar quantity of vinyl groups. The following quantities are reacted:

TABLE 1
Composition of the elastomer “Example 1”

| Components | Quantity (weight %) |
|---|---|
| Organohydrosiloxane H1 | 0.6 |
| Organohydrosiloxane H2 | 0.85 |
| Divinylsilane V1 | 2.1 |
| Divinylsilane V2 | 16.44 |
| Platinum(0)—1,2-divinyl— | 2 ppm (=0.015% of the |
| 1,1,3,3—tetramethyldisiloxan, | solution) |
| 0.1M in | |
| polydimethylsiloxane | |
| Dimethicone 5 cSt | 80 |

The vinyl components V1 and V2 are stirred into dimethicone and heated to 40° C. When the temperature is reached, the catalyst solution is added. When the catalyst solution is uniformly distributed, the organohydrosiloxanes H1 and H2 are added thereto. Stirring of the solution is continued and the reaction begins immediately. The end of the polymerization process is observed by means of gelling of the reaction mixture and a rapid increase in viscosity associated therewith. When the viscosity of the product no longer increases significantly, the reaction is concluded. This can take different lengths of time depending on the volumes of reaction mixture. The polymer concentration in the finished reaction product is 20%.

In order to complete the elastomer product “Example 1”, this is brought to a polymer concentration of 17.5% by addition of further dimethicone (5 cSt) and strong shearing. For this purpose, a corresponding quantity of reaction product is weighed into a suitable stirring vessel and—at this point without the addition of further dimethicone—sheared for 40 seconds using the L5M-A high-shear mixer from Silverson (using the “multi-purpose comminuting stator” and a rotary speed of 8000 rpm).

Subsequently, a corresponding quantity of dimethicone 5 cSt is added thereto, and is incorporated using the mixer. It is important to ensure uniform shearing of the whole product.

### EXAMPLE 2

### Preparation of Elastomer with Particularly Tack-Reducing Effects

The siloxane elastomer with particularly tack-reducing effects is prepared as described below:

A One organohydrosiloxane (e.g. Silmer H D5 (denoted H1, Siltech LLC, Lawrenceville, Ga., U.S.A.)) is reacted with

B one divinylsilane (e.g. Silmer Vin D5 (denoted V1, Siltech LLC, Lawrenceville, Ga., U.S.A.)) using the catalyst

C Platinum(0)-1,2-divinyl-1,1,3,3-tetramethyldisiloxane (e.g. as solution in polydimethylsiloxane (Sigma-Aldrich GmbH, Munich, Germany))

in the following manner, with dimethicone as solvent.

The quantities of the starting materials are selected such that the molar quantity of silane groups corresponds to the molar quantity of vinyl groups. The following quantities are reacted:

TABLE 2
Composition of the elastomer “Example 2”

| Components | Quantity (weight %) |
|---|---|
| Organohydrosiloxane H1 | 1.84 |
| Divinylsilane V1 | 18.15 |
| Platinum(0)—1,2-divinyl— | 2 ppm (=0.015% of the |
| 1,1,3,3—tetramethyldisiloxan, | solution) |
| 0.1M in | |
| polydimethylsiloxane | |
| Dimethicone 5 cSt | 80 |

The vinyl component V1 is stirred into dimethicone and heated to 40° C. When the temperature is reached, the catalyst solution is added. When the catalyst solution is uniformly distributed, the organohydrosiloxane H1 is added thereto. Stirring of the solution is continued and the reaction begins immediately. The end of the polymerization process is observed by means of gelling of the reaction mixture and a rapid increase in viscosity associated therewith. The polymer concentration in the finished reaction product is 20%.

In order to complete the elastomer product “Example 2”, this is brought to a polymer concentration of 17.5% by addition of further dimethicone (5 cSt) and strong shearing. For this purpose, a corresponding quantity of reaction product is weighed into a suitable stirring vessel with a corresponding quantity of dimethicone 5 cSt added thereto, and sheared for 120 seconds using the L5M-A high-shear mixer from Silverson (using the “multi-purpose comminuting stator” and a rotary speed of 8000 rpm).

TABLE 3
Examples 3-5: Emulsions with a content of
inventive siloxane elastomers

| | Example number | | |
|---|---|---|---|
| | 3 | 4 | 5 |
| PG-10 Stearate | | 0.5 | |
| Bis-(Glyceryl/Lauryl) Glyceryl Lauryl | | | 3 |
| Dimethicone (and) Caprylic/Capric | | | |
| Triglyceride (Abil EM 120) | | | |
| Sodium stearoyl glutamate | 0.2 | | |
| Cetyl stearyl alcohol | 1 | | |
| Caprylic/Capric Triglyceride | 1 | 5 | |
| Octyldodecanol | 5 | 5 | |
| Dimethicone | 8 | 5 | 7 |
| Ethylhexyl stearate | 3 | | 3 |
| Ethylhexyl methoxycinnamate | | 2 | |
| 2-Ethylhexyl 2-cyano-3-diphenylacrylate | | 2 | |
| (octocrylene) | | | |
| Ethylhexyltriazone | | 0.5 | |
| Butylmethoxydibenzoylmethane | | 1 | |
| Bis-ethylhexyloxyphenol- | | 1 | |
| methoxyphenyltriazine | | | |
| Ubiquinone (Q10) | | | 0.1 |
| Tocopheryl acetate | | | 0.5 |
| Methylpropanediol | 3 | | 2 |
| Trisodium EDTA | | 0.1 | |
| Glycerol | 5 | 10 | 3 |
| Panthenol | 0.5 | | |
| Phenoxyethanol | 0.5 | 0.5 | 0.3 |
| Denatured ethanol | 1 | 3 | |
| Xanthan Gum | 0.2 | | |
| Polyacrylic acid (carbomer) | 0.2 | 0.2 | |
| C10-30 alkyl acrylate crosspolymer | 0.2 | 0.2 | |
| Silicone elastomer according to example 1 | 50 | | 40 |
| Silicone elastomer according to example 2 | | 25 | |
| Fillers/additives (distarch phosphate, talc, | 2 | 1 | 1 |
| bentonite, silica, iron oxides) | | | |
| Perfume | q.s. | q.s. | q.s. |
| Water | to 100 | to 100 | to 100 |

TABLE 4
Compositions of the example formulations for
determining “in-vitro sand adhesion”

| INCI | Ex. 6 | Ex. 7 | Ex. 8 | Ex. 9 | Ex. 10 |
|---|---|---|---|---|---|
| Dimethicone | | 10 | | | |
| Dimethicone + Dimethicone | | | 10 | | |
| Crosspolymer (DC 9041, | | | | | |
| Dow Coming) | | | | | |
| Dimethicone + Dimethicone/ | | | | 10 | |
| Vinyl Dimethicone | | | | | |
| Crosspolymer (KSG-19, | | | | | |
| Shin-Etsu) | | | | | |
| Silicon elastomer (inventive | | | | | 10 |
| composition according to | | | | | |
| example 1) | | | | | |
| Sodium Stearoylglutamate | 0.3 | 0.3 | 0.3 | 0.3 | 0.3 |
| C12-15 alkyl benzoate | 2 | 2 | 2 | 2 | 2 |
| Glycerol + water | 7 | 7 | 7 | 7 | 7 |
| Methylpropanediol | 0.4 | 0.4 | 0.4 | 0.4 | 0.4 |
| Water + sodium hydroxide | 1.54 | 1.54 | 1.54 | 1.54 | 1.54 |
| solution | | | | | |
| Phenoxyethanol | 0.6 | 0.6 | 0.6 | 0.6 | 0.6 |
| Cetylsteatyl alcohol | 2 | 2 | 2 | 2 | 2 |
| Ammonium | 0.4 | 0.4 | 0.4 | 0.4 | 0.4 |
| Actyloyidimethyltaurate/VP | | | | | |
| Copolymer + water | | | | | |
| Acrylate/C10-30 Alkyl | 0.25 | 0.25 | 0.25 | 0.25 | 0.25 |
| Acrylate Crosspolymer | | | | | |
| Xanthan gum | 0.1 | 0.1 | 0.1 | 0.1 | 0.1 |
| Water | 67.5 | 57.5 | 57.5 | 57.5 | 57.5 |
| Alcoho + water | 0.4 | 0.4 | 0.4 | 0.4 | 0.4 |
| Water + Trisodum EDTA | 1 | 1 | 1 | 1 | 1 |
| Octocrylene | 8 | 8 | 8 | 8 | 8 |
| Butyl Methoxydibenzoyl- | 4.5 | 4.5 | 4.5 | 4.5 | 4.5 |
| methane | | | | | |
| Phenylbenzimidazole- | 4 | 4 | 4 | 4 | 4 |
| sulfonic acid | | | | | |

1.-4. (canceled)

5. A siloxane elastomer, wherein the siloxane elastomer is obtained by reacting
A one or more oligomeric organohydrosiloxanes comprising at least two SiH-bearing siloxane units per molecule of formula: 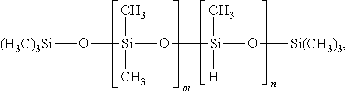
where m=0-40 and n=2-25; and
B one or more oligomeric divinylsilanes of formula: 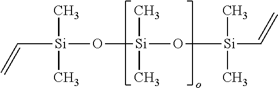
where o=5-90;
in the presence of
C platinum(0)-1,3-divinyl-1,1,3,3-tetramethyldisiloxane of formula: 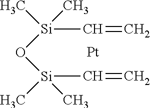
as hydrosilylation catalyst, wherein B is dissolved with stirring in a silicone and heated to a temperature ranging from 20° C. to 60° C., whereafter C is stirred in, followed by addition of A.

6. The siloxane elastomer of claim 5, wherein in the formula of A, m=0-30 and n=2-15.

7. The siloxane elastomer of claim 5, wherein in the formula of A, m=0-20 and n=4-10.

8. The siloxane elastomer of claim 5, wherein in the formula of B, o=10-80.

9. The siloxane elastomer of claim 5, wherein in the formula of B, o=15-70.

10. The siloxane elastomer of claim 5, wherein the silicone oil in which B is dissolved comprises dimethicone.

11. The siloxane elastomer of claim 5, wherein B dissolved in silicone oil is heated to a temperature ranging from 25° C. to 45° C.

12. The siloxane elastomer of claim 5, wherein a molar ratio A:B is from 10:1 to 1:10.

13. The siloxane elastomer of claim 5, wherein a molar ratio A:B is from 5:1 to 1:5.

14. The siloxane elastomer of claim 5, wherein a molar ratio A:B is from 2:1 to 1:2.

15. A cosmetic or dermatological preparation, wherein the preparation comprises one or more siloxane elastomers according to claim 5.

16. The preparation of claim 15, wherein the preparation comprises from 0.1% to 60% by weight of the one or more siloxane elastomers, based on a total weight of the preparation.

17. The preparation of claim 16, wherein the preparation comprises from 1% to 51% by weight of the one or more siloxane elastomers.

18. The preparation of claim 16, wherein the preparation comprises from 3% to 40% by weight of the one or more siloxane elastomers.

19. The siloxane elastomer of claim 5, wherein the siloxane elastomer is obtained by reacting
A one or more oligomeric organohydrosiloxanes comprising at least two SiH-bearing siloxane units per molecule of formula: 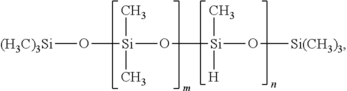
where m=0-30 and n=2-15; and
B one or more oligomeric divinylsilanes of formula: 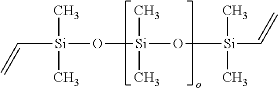
where o=10-80;
in the presence of
C platinum(0)-1,3-divinyl-1,1,3,3-tetramethyldisiloxane of formula: 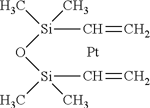
as hydrosilylation catalyst, wherein B is dissolved with stirring in a silicone and heated to a temperature ranging from 25° C. to 45° C., whereafter C is stirred in, followed by addition of A.

20. The siloxane elastomer of claim 19, wherein a molar ratio A:B is from 5:1 to 1:5.

21. The siloxane elastomer of claim 5, wherein the siloxane elastomer is obtained by reacting
A one or more oligomeric organohydrosiloxanes comprising at least two SiH-bearing siloxane units per molecule of formula: 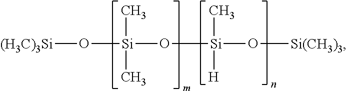
where m=0-20 and n=4-10; and
B one or more oligomeric divinylsilanes of formula: 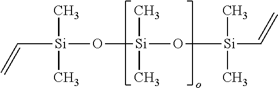
where o=15-70;
in the presence of
C platinum(0)-1,3-divinyl-1,1,3,3-tetramethyldisiloxane of formula: 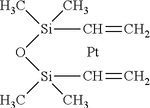
as hydrosilylation catalyst, wherein B is dissolved with stirring in a silicone comprising dimethicone and heated to a temperature ranging from 25° C. to 45° C., whereafter C is stirred in, followed by addition of A.

22. The siloxane elastomer of claim 21, wherein a molar ratio A:B is from 2:1 to 1:2.

23. A method of preparing a siloxane elastomer, wherein the method comprises reacting
A one or more oligomeric organohydrosiloxanes comprising at least two SiH-bearing siloxane units per molecule of formula: 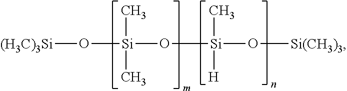
where m=0-40 and n=2-25; and
B one or more oligomeric divinylsilanes of formula: 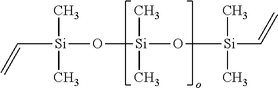
where o=5-90;
in the presence of
C platinum(0)-1,3-divinyl-1,1,3,3-tetramethyldisiloxane of formula: 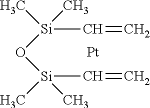
as hydrosilylation catalyst, wherein B is dissolved with stirring in a silicone and heated to a temperature ranging from 20° C. to 60° C., whereafter C is stirred in, followed by addition of A.

24. The method of claim 23, wherein a molar ratio A:B is from 10:1 to 1:10.
